(19) United States
(12) Patent Application Publication
(10) Pub. No.: US 2006/0083678 A1
(43) Pub. Date: Apr. 20, 2006

(54) Radio-labeled compounds, compositions, and methods of making the same

(76) Inventors: John V. Frangioni, Wayland, MA (US); Apara R. Dave, Franconia, NH (US); Daniel S. Kemp, Boston, MA (US)

Correspondence Address: FISH & RICHARDSON PC, P.O. BOX 1022, MINNEAPOLIS, MN, 55440-1022, US

(21) Appl. No.: 11/156,259

(22) Filed: Jun. 17, 2005

Related U.S. Application Data
(60) Provisional application No. 60/581,073, filed on Jun. 17, 2004.

(51) Int. Cl. A61K 51/00; C07D 309/30; A61M 36/14

(52) U.S. Cl. 424/1.11; 549/292

(57) ABSTRACT

¹⁸F radio-labeled compounds, methods of making the radio-labeled compounds, and applications of the same are disclosed.

### CROSS-REFERENCE TO RELATED APPLICATION

This application claims priority from U.S. Provisional Patent Application Ser. No. 60/581,073, filed on Jun. 17, 2004, which is incorporated herein by reference in its entirety.

### STATEMENT AS TO FEDERALLY SPONSORED RESEARCH

This invention was made with Government support under NIH Grant No. R21/R33CA88245. The Government thus has certain rights in the invention.

### TECHNICAL FIELD

This invention relates to radio-labeled compounds and compositions, and more particularly to ¹⁸F radio-labeled compounds and compositions, methods of making the radio-labeled compounds and compositions, and applications of the same.

### BACKGROUND

Positron emission tomography (PET) is useful for detection and imaging of cancer. Typically, a patient receives an intravenous injection an imaging agent, e.g., an ¹⁸F radio-labeled sugar, e.g., glucose. Once the imaging agent is distributed throughout the patient's body, a PET scanner detects the radio-labeled compound, and shows it as an image on a video screen. Typically, the images reveal information about chemistry taking place within organs being imaged. Although all cells use glucose, some cells, e.g., cancer cells, are more easily imaged then normal cells.

A common imaging agent is 2-deoxy-2-[¹⁸F]fluoro-D-glucose (¹⁸FDG), compound (1) in FIG. 1. A common method of synthesis of ¹⁸FDG is shown in FIG. 1. Synthesis starts with mannose triflate (A). After fluorination, producing 2-deoxy-2-[¹⁸F]fluoro-1,3,4,6-tetra-O-acetyl-D-glucose (B), and immobilization on a C18 column, base hydrolysis is used to remove the HOAc protective groups. Finally, C18 and neutral aluminum depletion chromatography are used to isolate the ¹⁸FDG (1).

Wide availability of PET imaging was hampered in the past because of a need for both dedicated PET imaging equipment and ¹⁸FDG (1), which has a short half-life (approximately 110 minutes). Several years ago, PET imaging was limited to research sites that were able to produce the ¹⁸F⁻ on-site with a cyclotron. Recently, an industry has been built to provide ¹⁸FDG (1) throughout the day to PET imaging facilities. Typically, ¹⁸FDG (1) is synthesized, and shipped regionally. In general, at least a half-life is consumed during synthesis and shipment. In some cases, it is possible to ship to sites which are two or more half-lives away. Its relative resistance to radiolysis facilitates production of ¹⁸FDG (1) in large quantities at high specific activity.

Although ¹⁸FDG (1) has been successful as a PET imaging agent, there is a need for new imaging agents. In particular, there is a need for imaging agents for cancers that are not ¹⁸FDG (1)-avid. Examples of cancers that are not ¹⁸FDG (1)-avid include bronchoalveolar cell cancer, lobular breast cancer, and some prostate cancers. There is also a general need to find more specific imaging agents which can enable better imaging.

### SUMMARY

In general, the invention is related to ¹⁸F radio-labeled compounds and compositions, methods of making the radio-labeled compounds and compositions, and applications of the same. We have discovered that ¹⁸FDG (1) can be converted into other radio-labeled compounds, e.g., conjugates with proteins, having a specific affinity for certain cancer cells, that can be useful in, e.g., in vivo pathology imaging, e.g., tumor imaging using PET.

Stable, but reactive intermediates can be produced from ¹⁸FDG (1) by oxidation of ¹⁸FDG (1) with an oxidant, prevention of lactone re-formation (re-cyclization) by protection at adjacent hydroxyl groups, and substitution of a carboxylic acid hydroxyl group with a leaving group (LG). The leaving group is sufficiently labile so that a conjugate can be easily formed with a nucleophilic moiety, e.g., a moiety that includes, e.g., an amino group, a hydroxyl group, or a thiol group, e.g., a protein, a protein fragment, a peptide, e.g., a low molecular weight peptide, a carbohydrate, or a polyol, e.g., polyethylene glycols, polypropylene glycols, and copolymers therefrom.

In one aspect, the invention features methods of making 2-deoxy-2-[¹⁸F]fluoro-D-glucose derivatives. The methods include oxidizing ¹⁸FDG (1) with an oxidant under first conditions and for a sufficient first time to produce a gluconic acid lactone (2) that is in equilibrium with its gluconic acid (3) form. The gluconic acid (3) form is protected by reacting two hydroxyl groups of the gluconic acid (3) form with a protecting moiety under second conditions and for a sufficient second time to prevent reversion of the gluconic acid (3) form to its gluconic acid lactone (2), and to produce a protected acid (4). The protected acid (4) has a carboxylic acid group that includes a carboxylic acid hydroxyl group. The carboxylic acid hydroxyl group of the protected acid (4) is substituted with a leaving group (LG), thereby forming an ¹⁸FDG derivative.

In some embodiments, the ¹⁸FDG derivatives are compounds of formula (5), where LG and R each, independently, includes an alkyl group, an alkenyl group, an alkynyl group, an aryl group, a heterocyclic group, a boron-containing group, or a mixture of such groups, and where LG and R each include no more than twenty carbon atoms.

The two reacted hydroxyl groups can be, e.g., located on adjacent carbon atoms, and the oxidant can be, e.g., diatomic bromine.

In some embodiments, the first conditions include using a buffer solution, e.g., a phosphate buffer; using water as a solvent; maintaining the pH from about 4 to about 9; maintaining the temperature between about 15 to about 50° C.; and maintaining the first time less than 3 hours.

In some embodiments, the second conditions include employing water as a solvent; maintaining a pH of about 0 to about 5 (e.g., 2, 3, or 4); maintaining a temperature from about 15 to about 60° C. (e.g., 25, 35, or 50); and maintaining the second time less than 3 hours (e.g., about 1 or 2 hours).

The two hydroxyl groups can be attached, e.g., to C5 and C6, or C4 and C5, or C4 and C6 of formula (3).

In some implementations, the protecting moiety is formaldehyde, dimethoxymethane, or boric acid. In some embodiments, the leaving group is O—N-succinimide.

In another aspect, the invention features compounds of formula (5), in which LG and R each, independently, includes an alkyl group, an alkenyl group, an alkynyl group, an aryl group, a heterocyclic group, a boron-containing group, or a mixture of such groups, and in which LG and R each comprise no more than twenty carbon atoms.

In some embodiments, LG is O—N-succinimide, and R is (CH₂)ₙ, n being an integer between 1 and 10, inclusive, e.g., n is between 1 and 5, inclusive, or n is between 1 and 3, inclusive. In a specific embodiment, LG is O—N-succinimide, and R is CH₂.

In another aspect, the invention provides compounds of formula (4), in which R includes an alkyl group, an alkenyl group, an alkynyl group, an aryl group, a heterocyclic group, a boron-containing group, or a mixture of such groups, and in which R includes no more than twenty carbon atoms.

In some embodiments, R is (CH₂)ₙ, n being an integer between 1 and 10, inclusive, e.g., n is between 1 and 5, inclusive, or n is between 1 and 3, inclusive.

In another aspect, the invention provides compounds of formula (10), (9), (8), (7), (6), (6′), (3), or (2).

In another aspect, the invention provides compositions including compounds of (10), (9), (8), (7), (6), (6′), (3), (2), or mixtures thereof.

In another aspect, the invention provides conjugates of formula (12′), in which R includes an alkyl group, an alkenyl group, an alkynyl group, an aryl group, a heterocyclic group or a mixture of such groups, and in which R includes no more than twenty carbon atoms, and in which R₁—NH₂ is a ligand or a targeting ligand comprising a protein, a protein fragment, a low molecular weight peptide, an antibody, a carbohydrate, an antigen, or a polymer.

In some embodiments, the targeting ligand is a low molecular weight peptide.

In another aspect, the invention features methods of imaging mammals, e.g., humans. The methods use any of the compounds disclosed herein.

In another aspect, the invention features methods of purifying radio-labeled 2-deoxy-2-[¹⁸F]fluoro-D-glucose derivatives. The methods include obtaining a composition comprising (¹⁸FDG) (1), a solvent, and a compound of formula (5), in which LG and R each, independently, includes an alkyl group, an alkenyl group, an alkynyl group, an aryl group, a heterocyclic group, a boron-containing group, or a mixture of such groups, and in which LG and R each comprise no more than twenty carbon atoms. The composition is passed through a column that includes an adsorbent. The absorbent binds to the compound of formula (5) with a greater affinity than other components of the composition. The compound of formula (5) is eluted, substantially free ¹⁸FDG (1).

In some embodiments, the compound of formula (5) is A¹⁸FDGA-NHS (8); the adsorbent is a resin, e.g., a crosslinked resin; the column, e.g., a disposable column, is sealed, and the solvent is water or an alcohol, e.g., ethanol.

In another aspect, the invention features methods of purifying a radio-labeled 2-deoxy-2-[¹⁸F]fluoro-D-glucose derivative. The methods include obtaining a composition including (¹⁸FDG) (1), a solvent, and a compound of formula (5), in which LG and R each, independently, includes an alkyl group, an alkenyl group, an alkynyl group, an aryl group, a heterocyclic group, a boron-containing group, or a mixture of such groups, and in which LG and R each include no more than twenty carbon atoms. The composition is passed through a column that includes an adsorbent. The absorbent binds with a greater affinity to components other than the compound of formula (5), allowing the compound of formula (5) to pass through the column at a faster rate than other components. The compound of formula (5) is collected, substantially free (¹⁸FDG) (1).

In general, advantages of the new methods and compositions include any one, or any combination, of the following. ¹⁸F radio-labeled compounds and compositions are provided using existing infrastructure, e.g., distribution channels and capital equipment, and are synthesized by starting with a readily available, relatively inexpensive, and radio-resistant moiety, ¹⁸FDG (1). The new compounds are made using proven chemistry and purification methods, and can have enhanced resistance to radiolysis. The new compounds can include a variety of moieties that can, for example, change polarity of the molecule and can, for example, enable rapid up-take by the body, and/or enable an easier and/or more efficient separation from other components of a reaction mixture.

The methods used for making the new compounds and compositions can provide a practitioner, e.g., a physician or a technician, with on-demand conversion that is convenient, cost-effective, reproducible, and that reduces the likelihood of human exposure to the radio-labeled compounds. When the new compounds and compositions are used as imaging agents, e.g., PET imaging agents, they can provide a more specific reagent to certain abnormal cells, e.g., cancer cells, and as a result, can provide better imaging of such abnormal cells. The new compounds and compositions can potentially provide earlier detection of the abnormal cells, thus saving lives.

The term “alkyl” denotes straight chain, branched, mono- or poly-cyclic alkyl moieties. Examples of straight chain and branched alkyl groups include methylene, alkyl-substituted methylene, methyl, ethyl, propyl, isopropyl, butyl, isobutyl, sec-butyl, t-butyl, amyl, isoamyl, sec-amyl, 1,2-dimethylpropyl, 1,1-dimethylpropyl, pentyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, octyl, 6-methylheptyl, 1-methylheptyl, 1,1,3,3-tetramethylbutyl, and the like. Examples of cyclic alkyl groups include cyclopropyl, cyclobutyl, cyclopentyl, cyclohexyl, cycloheptyl, cyclooctyl, cyclononyl, cyclodecyl, alkyl substituted ring systems, e.g., methylcycloheptyl, and the like.

The term “alkenyl” denotes straight chain, branched, mono- or poly-cyclic alkene moieties, including mono- or poly-unsaturated alkyl or cycloalkyl groups. Examples of alkenyl groups include vinyl, allyl, 1-methylvinyl, butenyl, iso-butenyl, 3-methyl-2-butenyl, 1-pentenyl, cyclopentenyl, 1-methylcyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl, 1,3,5,7-cycloocta-tetraenyl, and the like.

The term “alkynyl” denotes straight chain, branched, mono- or poly-cyclic alkynes. Examples of alkynyl groups include ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl, 5-hexynyl, 10-undecynyl, 4-ethyl-1-octyn-3-yl, and the like.

The term “aryl” denotes single, polynuclear, conjugated, or fulsed residues of aromatic hydrocarbons. Examples of aryl include phenyl, biphenyl, phenoxyphenyl, naphthyl, tetahydronaphthyl, anthracenyl, and the like.

The term “heterocyclic” denotes mono- or poly-cyclic heterocyclic groups containing at least one heteroatom selected from nitrogen, phosphorus, sulphur, silicon, and oxygen. Examples of heterocyclic groups include pyrrolyl, pyrrolinyl, imidazolyl, pyrazolyl, pyridyl, pyrimidinyl, pyrazinyl, pyridazinyl, triazolyl, pyrrolidinyl, imidazolidinyl, piperdino or piperazinyl, indolyl, isoindolyl, indolizinyl, benzimidazolyl, quinolyl, isoquinolyl, indazolyl, benzotriazolyl, tetrazolopyridazinyl, pyranyl, and the like.

The alkyl, alkenyl, alkynyl, aryl, or hetercyclic groups may be optionally substituted with a heteroatom, e.g., nitrogen, phosphorus, sulphur, silicon, or oxygen atoms, and can be substituted with functional groups containing the heterotom, e.g., carbonyl groups.

The term “protein” denotes a moiety that comprises a plurality of amino acids, covalently linked by peptide bonds. Proteins can be, for example, found in nature, or they can be synthetic equivalents of those found in nature, or they can be synthesized, non-natural proteins. In addition to amino acids, a protein can include other moieties, e.g., moieties that include sulfur, phosphorous, iron, zinc and/or copper, along its backbone. Proteins can, for example, also contain carbohydrates moieties, lipid moieties, and/or nucleic acid moieties. Specific examples of proteins include keratin, elastin, collagen, hemoglobin, ovalbumin, casein, and hormones, actin, myosin, annexin V, and antibodies. As used herein, the terms “polypeptide” and “protein” are used interchangeably, unless otherwise stated.

The term “antibody” as used herein refers to an immunoglobulin molecule or immunologically active portion thereof, i.e., an antigen-binding portion.

The antibody can be a polyclonal, monoclonal, recombinant, e.g., a chimeric, de-immunized or humanized, fully human, non-human, e.g., murine, or single chain antibody. In some embodiments the antibody has effector function and can fix complement. In some embodiments, the antibody has reduced or no ability to bind an Fc receptor. For example, the antibody can be an isotype or subtype, fragment or other mutant, which does not support binding to an Fc receptor, e.g., it has a mutagenized or deleted Fc receptor binding region. The antibody can be coupled to a toxin or imaging agent.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a general synthetic method that illustrates making 2-deoxy-2-[¹⁸F]fluoro-D-glucose, ¹⁸FDG (1), from mannose triflate (A).

FIG. 2 is a general synthetic method that illustrates oxidizing ¹⁸FDG (1), protecting ¹⁸FDGluconic acid (3) to prevent reversion to ¹⁸FDGluconic acid lactone (2), and substituting the carboxylic acid OH of the resulting protected ¹⁸FDGA (4) with a leaving group (LG), resulting in ¹⁸FDGA-LG (5).

FIG. 3 is a specific embodiment that illustrates the synthetic method shown in FIG. 2 in which an NHS ester (8) is formed, and in which R is —CH₂—.

FIG. 4 shows in detail formation of an acetal-protected moiety A¹⁸FDGA (7) from ¹⁸FDGluconic acid (3).

FIGS. 5 and 5A are schematics that illustrate a method of purifying ¹⁸FDGA-LG (5) that minimizes time required, and human exposure.

FIG. 6 is a schematic of a method of making conjugates.

FIG. 7 is a representation of potential ligands that can be used to make conjugates.

FIG. 8 is a schematic that illustrates a method of purifying conjugates.

FIG. 9A is a mass spectrum which shows (2), (2)+NH₄⁺, (3), and (3)+NH₄⁺.

FIG. 9B is a mass spectrum which shows (1)+NH₄⁺.

FIG. 10A is an HPLC trace of a region that includes (2)+(3), and a region that includes (8).

FIG. 10B is an HPLC trace that includes only (2)+(3).

FIGS. 11A-11C are a CT data set, a PET data set, and a micro-CT data set, respectively.

FIG. 11D is a data set generated by co-registration.

Like reference symbols in the various drawings indicate like elements.

### DETAILED DESCRIPTION

In general, ¹⁸F radio-labeled compounds and compositions, methods of making the radio-labeled compounds and compositions, and applications of the same are disclosed herein. We have discovered that 2-deoxy-2-[¹⁸F]fluoro-D-glucose, ¹⁸FDG (1), can be converted into stable, but reactive intermediate compounds, i.e., ¹⁸FDG derivatives. ¹⁸FDG derivatives can be converted to conjugates, e.g., by reaction of an ¹⁸FDG derivative with, e.g., a nucleophilic moiety, e.g., a moiety that includes, an amino group, a hydroxyl group, or a thiol group, e.g., a protein, a protein fragment, a peptide, e.g., a low molecular weight peptide, or a carbohydrate. The new conjugates have a specific affinity for certain abnormal cells, e.g., cancer cells, and can be useful in, e.g., in vivo pathology imaging, e.g., tumor imaging using PET.

Methodology for Synthesizing Stable Radio-Labeled ¹⁸FDG Derivatives

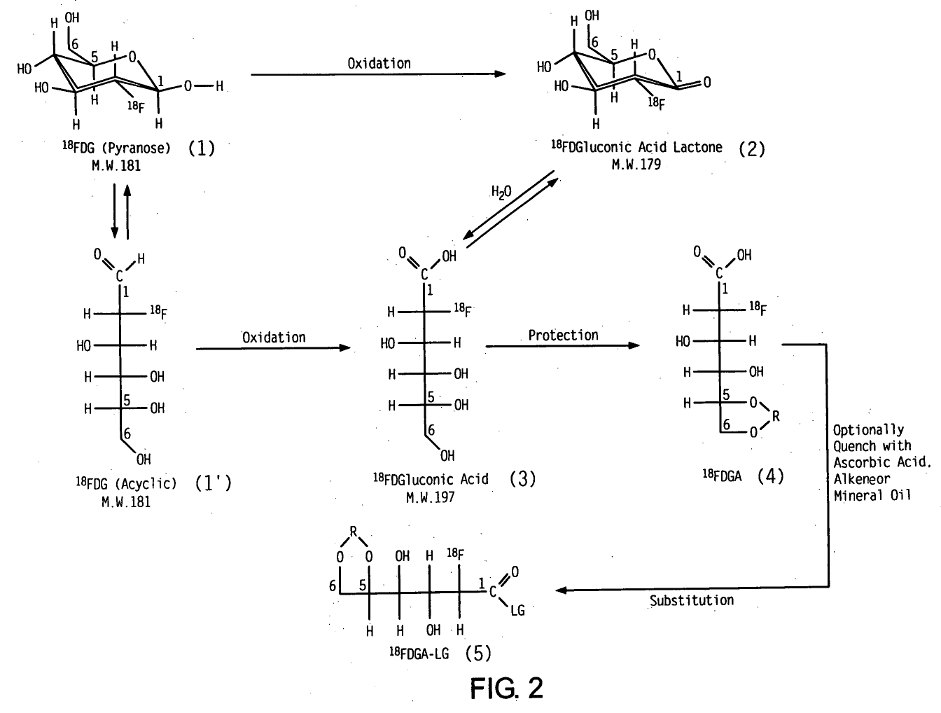

Referring to FIG. 2, a synthetic route for producing radio-labeled ¹⁸FDG derivatives includes oxidation of ¹⁸FDG (1) with an oxidant, e.g., diatomic bromine, prevention of lactone re-formation (re-cyclization) by protection, e.g., acetal protection, at adjacent hydroxyl groups, e.g., attached at C5 and C6, and substituting a carboxylic acid hydroxyl group on C1 with a leaving group (LG).

More specifically, a method of making a radio-labeled ¹⁸FDG derivative includes oxidizing ¹⁸FDG (1) with an oxidant, e.g., diatomic bromine, under first conditions and for a sufficient first time to produce a gluconic acid lactone (2) that is in equilibrium with its gluconic acid (3) form. The gluconic acid form (3) is protected by reacting two adjacent hydroxyl groups, e.g., at C5 and C6, of the gluconic acid (3) form with a protecting moiety, e.g., formaldehyde, to prevent reversion of the gluconic acid (3) form to its gluconic acid lactone form (2). The reacting of the two adjacent hydroxyl groups, e.g., at C4 and C5, or C5 and C6, or C3 and C5, of the gluconic acid (3) form with the protecting moiety occurs under second conditions and for a sufficient second time to produce a protected acid (4). The protected acid (4) has a carboxylic acid group that includes a carboxylic acid hydroxyl group. The carboxylic acid hydroxyl group of the protected acid (4) is substituted with a leaving group (LG), thereby forming a compound of formula (5). The skilled artisan will understand that ¹⁸FDG (1) is in equilibrium with its acyclic aldehyde form ¹⁸FDG (acyclic) (1′).

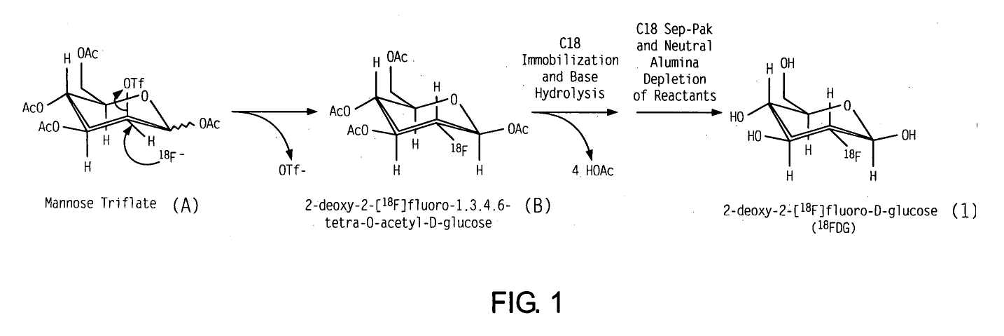

Major U.S. suppliers for 2-deoxy-2-[¹⁸F]fluoro-D-glucose, ¹⁸FDG (1), include Cardinal Health, also known as Syncor, and PETnet. Both suppliers make the ¹⁸FDG (1) by fluorination of mannose triflate (A), base hydrolysis of the resulting intermediate (B), and chromatographic depletion to yield pure ¹⁸FDG (1) product, as shown in FIG. 1. Typically, a standard clinical dose is about 10-20 mCi. It appears that material from both suppliers is quite similar. Analysis of material obtained from Cardinal Health and PETnet is shown below in TABLE 1. As shown in TABLE 1, PETnet makes no adjustment for tonicity, while Cardinal Health supplies the material in saline.

TABLE 1
Analysis of ¹⁸FDG (1)

| SUPPLIER | Cardinal Health | PETnet |
|---|---|---|
| CONCENTRATION (1) | 55 nM (10 mCi) | 55 nM (10 mCi) |
| CONCENTRATION NaCl | 150 nM | 0 |
| PH | 4.5-7.0 | 4.5-7.5 |

Suitable oxidants include, for example, diatomic chlorine, diatomic bromine, iodine, hypochlorite, e.g., sodium hypochorite, permanganate, e.g., potassium permanganate, hydrogen peroxide, organic peroxides, e.g., benzoyl peroxide, and metals in a high oxidation state, e.g., Cr(VI).

The first conditions can include, e.g., a buffer solution, e.g., a phosphate buffer. The first conditions can also include, e.g., employing water as a solvent, maintaining a pH of from about 4 to about 10, e.g., from about 6 to about 8, and maintaining a temperature from about 0 to about 50° C., e.g., from about 20 to about 30 ° C. For example, when a concentration of the oxidant is about 1 to about 400 mM, e.g., from about 50 to about 100 mM, a concentration of ¹⁸FDG (1) is about 0.5 to about 10 mM, e.g., from about 1 to about 5 mM, and the temperature of an aqueous solution is maintained at about 20 to about 30° C., oxidation of ¹⁸FDG (1) to ¹⁸FDGluconic acid lactone (2) is generally complete after 0 to about 6 hours.

The gluconic acid form (3) is protected by reacting two adjacent hydroxyl groups, e.g., at C5 and C6, of the gluconic acid (3) form with a protecting moiety. Referring particularly to formula (4) of FIG. 2, R can be a moiety that includes an alkyl group, an alkenyl group, an alkynyl group, an aryl group, a heterocyclic group, a boron-containing group, or a mixture of such groups. The moiety can include twenty carbon atoms or less. In some embodiments, R is (CH₂)ₙ, where n is an integer between I and 10, inclusive, e.g., between 1 and 5, inclusive, e.g., between 1 and 3, inclusive. In a particular embodiment, the protecting moiety is formaldehyde, dimethoxymethane, or boric acid. When the protecting moiety is dimethoxymethane, R is CH₂.

The second conditions can include, e.g., a buffer solution, e.g., a phosphate buffer. The second conditions can also include, e.g., employing water as a solvent, maintaining a pH of from about 0 to about 6, e.g., from about 1 to about 3, and maintaining a temperature from about 15 to about 60° C., e.g., from about 30 to about 40° C. For example, when a concentration of formaldehyde is about 0.1 to about 1.5 M, e.g., 0.7 to about 1 M, a concentration of lactone (2) and acid (3) combined is about 1 to about 20 mM, e.g., from about 5 to about 10 mM, a temperature of an aqueous solution is maintained at about 30 to about 40° C., and a pH is about 1 to about 3, protection of acid (3), forming (5) is generally complete after 0 to about 5 hours, e.g., 1 to about 2 hours.

The carboxylic acid hydroxyl group of the protected acid (4) is substituted with a leaving group (LG). The leaving group (LG) is a moiety that includes an alkyl group, an alkenyl group, an alkynyl group, an aryl group, a heterocyclic group, a boron-containing group, or a mixture of such groups. The moiety includes twenty carbon atoms or less. For example, the leaving group (LG), together with the adjacent carbonyl group, can be an ester, e.g., an N-hydroxysuccinimide (NHS) ester, or a substituted NHS ester, an amide, or a thioester. For example, the leaving group can be Woodward's reagent K or N-ethyl-3-phenylisxazolium-3′-sulfonate. Generally, LG⁻ is a weaker base than OH⁻, or put another way, LG-H is a stronger acid than water. LG-H has, for example, a pKA or less than 35 when measured in DMSO, e.g., 30, 28, 24, 22, 20, 18, 14, 13, 11, 10, 8, 7 or less, e.g., 5. pKa values for various organic moieties have been tabulated by Bordwell, see, for example, Bordwell et al., Accts. Chem. Research 21, 456 (1988).

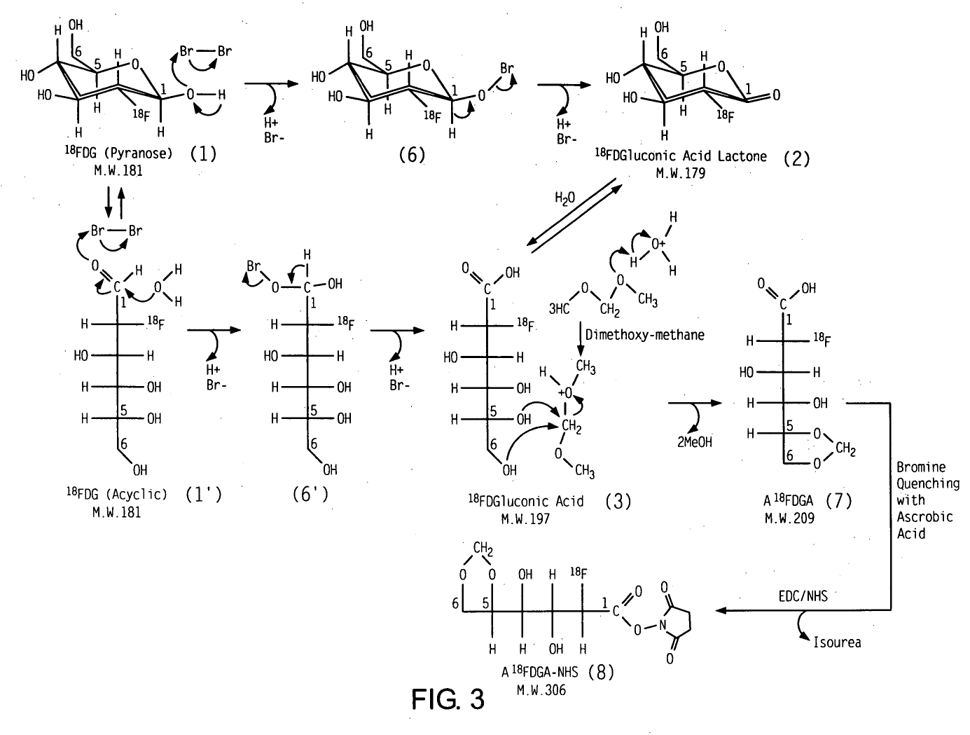

FIG. 3 shows a specific embodiment in which an NHS ester, A¹⁸FDGA-NHS (8), is formed from 5,6-acetal-2-deoxy-2-[¹⁸F]fluorogluconic acid, A¹⁸FDGA (7), utilizing a “one-pot” synthetic strategy. “One-pot” means that intermediates, e.g., (2), (6′) and (7), are not separated and purified during synthesis, but rather all the reactions leading up to product (8) are carried out in a single vessel. This is desirable because of the relatively short half-life of the radio-labeled compounds, and it is also a way to minimize human exposure to the radio-labeled compounds. Briefly, the synthesis includes oxidation of ¹⁸FDG (1) with bromine, prevention of lactone (2) re-formation by acetal protection at C5 and C6, quenching excess bromine with ascorbic acid, and forming the NHS ester (8) using EDC, 1-ethyl-3-[3-dimethylamino-propyl]carbodiimide hydrochloride.

As shown in FIG. 3, pyranose (cyclic) (1) and acyclic (1′) forms of ¹⁸FDG are in equilibrium in aqueous solutions, but the cyclic form is greatly favored (typically >99% of the total). Based on a calculated specific activity of 10 mCi of ¹⁸FDG (1) in a standard 10 ml dose, a chemical concentration of ¹⁸FDG (1) is approximately 55 nM. The addition of 10 mM bromine to a phosphate buffer solution of ¹⁸FDG (1) results in oxidation at C1, producing lactone (2). The reaction is should be completed within about 5-10 minutes with approximately a 96% yield. As can be seen from FIG. 3, acid (3) and lactone (2) are also in equilibrium. Approximately 50% of each form is present in solution at pH 7.0.

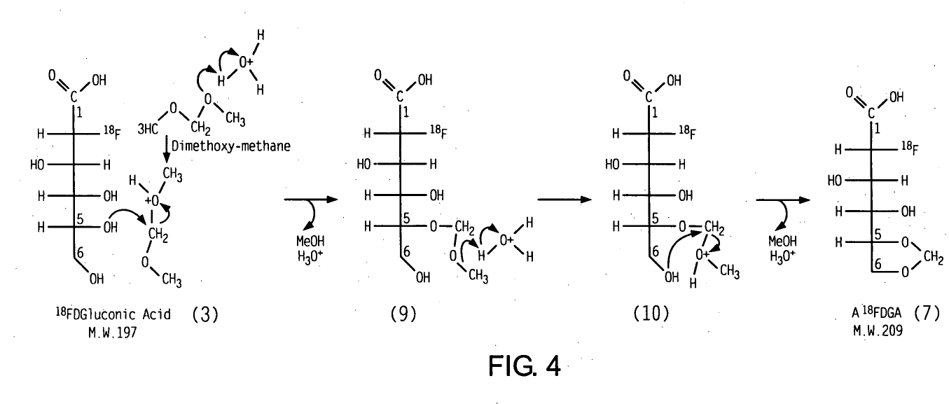

Referring now to FIGS. 3 and 4, to prevent re-formation of lactone (2), C5 and C6 are protected with an acetal group, using dimethoxymethane as the protecting moiety. Briefly, dimethoxymethane is reacted at equimolar concentrations with ¹⁸FDGluconic acid (3) in the presence of hydrobromic acid. Two sequential attacks on dimethoxymethane by hydroxyl groups attached to C5 and C6 of ¹⁸FDGluconic acid (3), produces intermediates (9) and (10). Cylization of intermediate (10), produces acetal A¹⁸FDGluconic acid (7). This reaction should be complete within minutes at room temperature.

Adding a two-fold molar excess of ascorbic acid quenches excess bromine. The quenching reaction should be complete within about ten minutes at room temperature. Ascorbic acid has an advantage of being soluble in aqueous environments.

In other embodiments, a hydrocarbon, e.g., containing alkyl or alkenyl groups, e.g., a mineral oil, is used as the quenching agent. A hydrocarbon can be advantageous since a two phase system results that can be easier to separate. In still other embodiments no excess oxidant is used, so no quenching agent is used.

After excess bromine is quenched, a succinimidyl ester (8) is formed at the free carboxylic acid using, e.g., 1-ethyl-3-[3-dimethylaminopropyl]carbodiimide hydrochloride (EDC), and N-hydroxysuccinimide (NHS), both being available from Aldrich Chemical. The reaction mixture is pH adjusted so that a pH of the reaction mixture is approximately 5.5. This is done by addition of 50 mM sodium phosphate buffer. EDC and NHS are added from concentrated stocks to a final concentration of 10 mM each (greater that a twenty-fold molar excess relative to the carboxylic acid (7)). After reaction for 1 hour at room temperature, conversion of (7) to (8) should be nearly complete. In some embodiments, the NHS ester is formed by heating to 150° C. for 1-3 minutes.

Purification of Radio-Labeled ¹⁸FDG Derivatives

Briefly, a closed-system purification strategy that utilizes a column, e.g., a one-time use disposable column, to purify ¹⁸FDG radio-labeled derivatives, e.g., an acetal-protected succinimidyl ester, e.g., A¹⁸FDGA-NHS (8), is often desirable because of the relatively short half-lives of the radio-labeled compounds, and also because it minimizes human exposure to the radio-labeled compounds. Often reaction mixtures are complex, containing, for example, salts, e.g., sodium chloride and phosphates, EDC, NHS, ascorbic acid, some unreacted oxidant, e.g., bromine, and unreacted ¹⁸FDG (1).

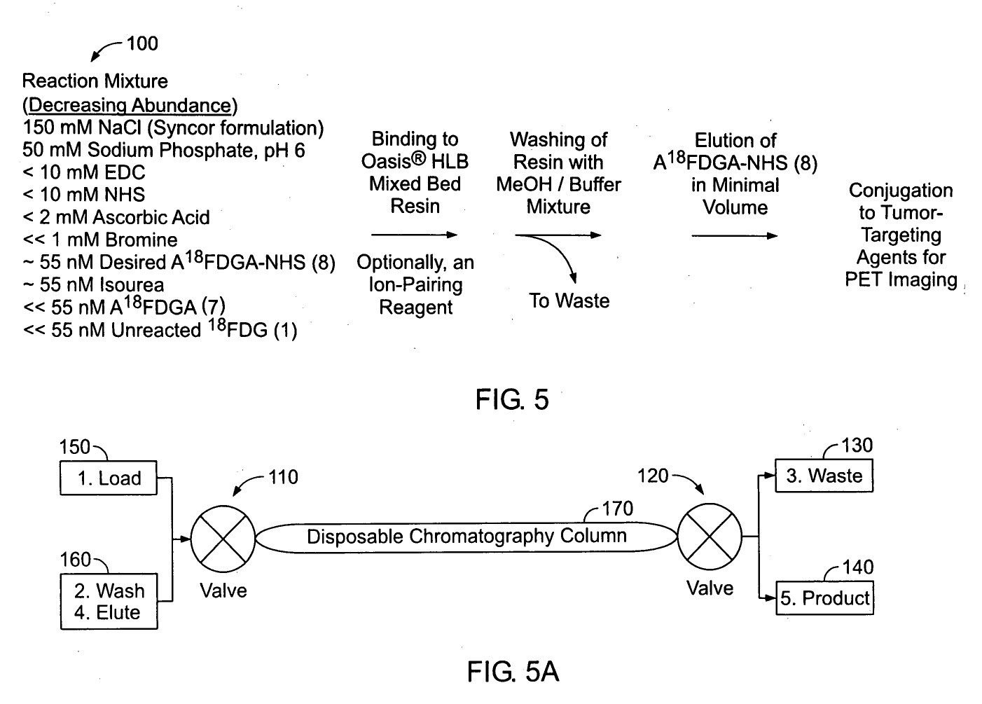

Referring to FIGS. 5A and 5B, a specific embodiment for purifying A¹⁸FDGA-NHS (8), is shown. The method shown for purification of A¹⁸FDGA-NHS (8) can, for example, also be applied generally to compounds of formula (5). As shown in FIG. 5A, at an end of the synthetic procedure shown in FIG. 3, the reaction is a complex mixture that consists of multiple salts, unwanted derivatives, reagents and starting materials. A¹⁸FDGA-NHS (8) can, for example, be purified from the unwanted components of the single pot reaction mixture by passing the reaction mixture through a disposable column containing an adsorbent, e.g., a polymeric resin, e.g., a cross-linked copolymer of m-divinylbenzene and N-vinylpyrrolidone. Suitable disposable columns are, e.g., Oasis® sample extraction columns, that are hydrophilic-lipophilic-balanced, and available from Waters, Milford, Mass. ¹⁸FDG (1) and A¹⁸FDGA (7) should interact only slightly with the adsorbent of the disposable column, relative to A¹⁸FDGA-NHS (8), the desired product of the reaction. In addition, salts, EDC, NHS, ascorbic acid, and isourea are not expected to interact strongly with the adsorbent of the disposable column. As a result, unwanted materials elute relatively quickly through the disposable column, leaving A¹⁸FDGA-NHS (8) adsorbed on the column. A¹⁸FDGA-NHS (8) can be eluted from the column with a solvent less polar than water, e.g., acetonitrile.

A¹⁸FDGA-NHS (8) is synthesized, e.g., in a Luer-lock syringe. If the reaction is carried out in a Luer-lock® syringe, the syringe will include a reaction mixture 1.00 at an end of the reaction period. Reaction mixture 100 includes unwanted components, as well as the desired product, A¹⁸FDGA-NHS (8). At the end of the reaction period, mixture 100 is diluted with water and 0.1% trifluoroacetate (TFA) to a volume of 5 ml. A 2.1×20 mm Oasis®-HLB column that includes 5 sum diameter resin beads (Waters Catalog #186002034) is inserted into its column holder, and three-way Luer-lock® stop-cocks 110, 120 are connected to both ends of a column 170. Outflow stop-cock 120 is connected separately to a waste vial 130, and a collection vial 140 which is used for collecting the desired product. Inflow stop-cock 110 is connected separately to a reaction syringe 150, and to a washing/elution syringe 160 containing a desired concentration of eluant, e.g., H₂O/acetonitrile+0.1% TFA. With inflow stop-cock 110 turned to reaction syringe 150, and outflow stop-cock 120 turned to waste vial 130, reaction mixture 100 is loaded onto column 170 that optionally contains an ion-pairing agent, e.g., Waters PIC® reagents, and then reaction syringe 150 is replaced with a 30 cc wash syringe 160 containing an appropriate ratio of H₂O/acetonitrile+0.1% TFA, for example, 50:50 H₂O/acetonitrile+0.1% TFA. Column 170 is washed with at least 20 column volumes to remove undesired reactants, and then inflow stop-cock 110 turned to the elution syringe 160, and outflow stop-cock 120 is turned to collection vial 140. The eluant, containing the desired product is collected, and optionally analyzed, e.g., by HPLC, and/or mass spectroscopy, e.g., after freezing with liquid nitrogen and allowing overnight decay.

A consideration in developing ¹⁸FDG (1) conversion and purification strategies is an amount of time involved in each step relative to the half-life of ¹⁸F (approximately 110 minutes). Many of the chemical transformations shown in FIG. 3 are rapid, and are typically complete in minutes. For example, bromine oxidation, and acetal formation at C5 and C6 should take less than about ten minutes to complete. Likewise, quenching of unreacted bromine will likely require no more than ten minutes. The most time consuming step is synthesis of the NHS ester (8) by EDC coupling, which takes about one hour at room temperature. However, using excess, e.g., a twenty-fold excess, of EDC and NHS relative to (7), can reduce reaction time needed to generate the NHS ester (8). Column purification, as discussed above, can take up to twenty minutes to complete. Conversion and purification steps should be done in less than one half-life of ¹⁸F, e.g., less than 110 minutes.

Methodology for Synthesizing Conjugates of ¹⁸FDGA (5)

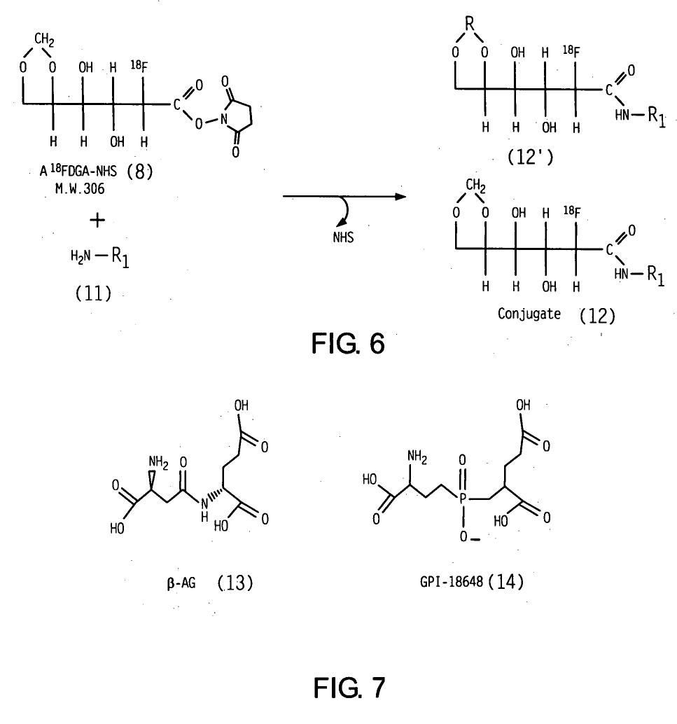

Referring to FIGS. 2 and 6, a compound of formula (5), e.g., A¹⁸FDGA-NHS (8), can be reacted with ligands, e.g., targeting ligands, to create novel conjugate imaging agents, e.g., for in vivo PET imaging. Such conjugates can provide, e.g., a more specific reagent for abnormal cells, e.g., cancer cells, and as a result, can provide better imaging of such abnormal cells. The new conjugates can potentially provide earlier detection of the abnormal cells, thus saving lives. In general, a conjugate, e.g., (12), or more generally (12′), is formed by reaction between a compound of formula 5, e.g., (8), and a nucleophilic moiety that includes a nucleophilic portion. R₁—NH₂ of (12), or (12′) can be, for example, a protein, a protein fragment, a peptide, e.g., octreotide (sandostatin), a low molecular weight peptide, an antibody, a carbohydrate, or an antigen. The nucleophilic portion can be, for example, a primary amine group, a thiol group, or a hydroxyl group. Possible proteins, protein fragments, low molecular weight peptides, antibodies, carbohydrates, or antigens can be found in G. Hermanson, Bioconjugate Techniques: Academic Press (November 1995, ISBN 012342335X).

Protein Targeting Ligands

A specific protein useful for preparing such a conjugate is annexin V, which is capable of binding with high affinity to the phosphatidylserine exposed during either apoptosis or necrosis of cells.

Antibody Targeting Ligands

A compound of formula (5), e.g., A¹⁸FDGA-NHS (8), can be also reacted with an antibody to create novel conjugate imaging agents with enhanced specificity, e.g., for in vivo PET imaging. Specific examples of antibodies are monoclonal antibodies to 10 prostate-specific membrane antigen (PSMA), e.g., 7E11-C5.3 antibody. Typically, antibodies and antibody fragments have a molecular weight of greater than about 30,000 Daltons.

A number of antibodies against cancer-related antigens are known; exemplary antibodies are described in TABLES 2-3 (Ross et al., Am J Clin Pathol 119(4):472-485, 2003).

TABLE 2
Approved Anticancer Antibodies

| | | | | Approved and |
|---|---|---|---|---|
| | Source | | | Investigational |
| Drug Name | (Partners)* | Type | Target | Indications |
| Alemtuzumab | BTG, West | Monoclonal | CD52 | Chronic |
| (Campath) | Conshohocken, | antibody, | | lymphocytic and |
| | PA (ILEX | humanized; | | chronic |
| | Oncology, | anticancer, | | myelogenous |
| | Montyille, NJ; | immunologic; | | leukemia; multiple |
| | Schering AG, | multiple sclerosis | | sclerosis, chronic |
| | Berlin, Germany) | treatment; | | progressive |
| | | immunosuppressant | | |
| Daclizumab | Protein Design | Monoclonal IgG1 | IL-2 receptor, | Transplant |
| (Zenapax) | Labs, Fremont, | chimeric; | CD25 | rejection, general |
| | CA (Hoffmann- | immunosuppressant; | | and bone marrow; |
| | La Roche, | antipsoriatic; | | uveitis; multiple |
| | Nutley, NJ) | antidiabetic; | | sclerosis, relapsing- |
| | | ophthalmologic; | | remitting and |
| | | multiple sclerosis | | chronic progressive; |
| | | treatment | | cancer, leukemia, |
| | | | | general; psoriasis; |
| | | | | diabetes mellitus, |
| | | | | type 1; asthma; |
| | | | | ulcerative colitis |
| Rituximab | IDEC | Monoclonal IgG1 | CD20 | Non-Hodgkin |
| (Rituxan) | Pharmaceuticals, | chimeric; | | lymphoma, B-cell |
| | San Diego, CA | anticancer, | | lymphoma, chronic |
| | (Genentech, | immunologic; | | lymphocytic |
| | South San | antiarthritic, | | leukemia; |
| | Francisco, CA; | immunologic; | | rheumatoid |
| | Hoffmann-La | immunosuppressant | | arthritis; |
| | Roche; Zen-yaku | | | thrombocytopenic |
| | Kogyo, Tokyo, | | | purpura |
| | Japan) | | | |
| Trastuzumab | Genentech | Monoclonal IgG1 | p185neu | Cancer: breast, non- |
| (Herceptin) | (Hoffmann-La | humanized; | | small cell of the |
| | Roche; | anticancer, | | lung, pancreas |
| | ImmunoGen, | immunologic | | |
| | Cambridge, MA) | | | |
| Gemtuzumab | Wyeth/AHP, | Monoclonal IgG4 | CD33/cali- | Acute myelogenous |
| (Mylotarg) | Collegeville, PA | humanized | cheamicin | leukemia (patients |
| | | | | older than 60 y) |
| Ibritumomab | IDEC | Monoclonal IgG1 | CD20/yttrium | Low-grade |
| (Zevalin) | Pharmaceuticals | murine; anticancer | 90 | lymphoma, |
| | | | | follicular |
| | | | | lymphoma, |
| | | | | transformed non- |
| | | | | Hodgkin lymphoma |
| | | | | (relapsed or |
| | | | | refractory) |
| Edrecolomab | GlaxoSmithKline, | Monoclonal IgG2A | Epithelial cell | Cancer: colorectal |
| (Panorex) | London, England | murine; anticancer | adhesion | |
| | | | molecule | |

TABLE 3
Selected Anticancer Antibodies in Clinical Trials

| | | | |
|---|---|---|---|
| Clinical Trial | | | Investigational |
| Status/Drug Name | Source | Features | Indications |

Phase 3

| | | | |
|---|---|---|---|
| Tositumomab | Corixa, Seattle, WA | Anti-CD20 murine | Non-Hodgkin |
| (Bexxar) | | monoclonal antibody | lymphoma |
| | | with iodine 131 | |
| | | conjugation | |
| CeaVac | Titan | Anti-CEA murine | Cancer: colorectal, |
| | Pharmaceuticals, | monoclonal antibody; | non-small cell of |
| | South San | anticancer | the lung, breast, |
| | Francisco, CA | immunologic vaccine | liver |
| Epratuzumab | Immunomedics, | Chimeric monoclonal | Non-Hodgkin |
| (LymphoCide) | Morris Plains, NJ | antibody; anticancer | lymphoma |
| | | immunologic; | |
| | | immunosuppressant | |
| Mitumomab | ImClone Systems, | Murine monoclonal | Small cell cancer of |
| | New York, NY | antibody; anticancer | the lung; melanoma |
| | | immunologic | |
| Bevacizumab | Genentech, South | Anti-VEGF | Cancer: colorectal, |
| (Avastin) | San Francisco, CA | humanized | breast, non-small |
| | | monoclonal antibody; | cell of the lung; |
| | | anticancer | diabetic retinopathy |
| | | immunologic; | |
| | | antidiabetic; | |
| | | ophthalmologic | |
| Cetuximab (C-225; | ImClone Systems | Anti-EGFR chimeric | Cancer: head and |
| Erbitux) | | monoclonal antibody; | neck, non-small cell |
| | | anticancer | of the lung, |
| | | immunologic | colorectal, breast, |
| | | | pancreas, prostate |
| Edrecolomab | Johnson & Johnson, | Murine monoclonal | Cancer: colorectal |
| | New Brunswick, NJ | antibody; anticancer | and breast |
| | | immunologic | |
| Lintuzumab | Protein Design | Chimeric monoclonal | Acute myelogenous |
| (Zamyl) | Labs, Fremont, CA | antibody; anticancer | leukemia; |
| | | immunologic | myelodysplastic |
| | | | syndrome |
| MDX-210 | Medarex, Princeton, | Bispecific chimeric | Cancer: ovarian, |
| | NJ; Immuno- | monoclonal antibody; | prostate, colorectal, |
| | Designed | anti-HER-2/neu-anti- | renal, breast |
| | Molecules, Havana, | Fc gamma RI; | |
| | Cuba | anticancer | |
| | | immunologic | |
| IGN-101 | Igeneon, Vienna, | Murine monoclonal | Cancer: non-small |
| | Austria | antibody; anticancer | cell of the lung, |
| | | immunologic | liver, colorectal, |
| | | | esophageal, |
| | | | stomach |

Phase 2

| | | | |
|---|---|---|---|
| MDX-010 | Medarex | Humanized anti- | Cancer: prostate, |
| | | HER-2 monoclonal | melanoma; |
| | | antibody; anticancer | infection, general |
| | | immunologic; | |
| | | immunostimulant | |
| MAb, AME | Applied Molecular | Chimeric monoclonal | Cancer: sarcoma, |
| | Evolution, San | antibody; anticancer | colorectal; |
| | Diego, CA | immunologic; | rheumatoid arthritis; |
| | | imaging agent; | psoriatic arthritis |
| | | antiarthritic | |
| | | immunologic; | |
| | | ophthalmologic; | |
| | | cardiovascular | |
| ABX-EGF | Abgenix, Fremont, | Monoclonal | Cancer: renal, non- |
| | CA | antibody, human; | small cell of the |
| | | anticancer | lung, colorectal, |
| | | immunologic | prostate |
| EMD 72 000 | Merck KGaA, | Chimeric monoclonal | Cancer: stomach, |
| | Darmstadt, | antibody; anticancer | cervical, non-small |
| | Germany | immunologic | cell of the lung, |
| | | | head and neck, |
| | | | ovarian |
| Apolizumab | Protein Design Labs | Chimeric monoclonal | Non-Hodgkin |
| | | antibody; anticancer | lymphoma; chronic |
| | | immunologic | lymphocytic |
| | | | leukemia |
| Labetuzumab | Immunomedics | Chimeric monoclonal | Cancer: colorectal, |
| | | antibody; | breast, small cell of |
| | | immunoconjugate; | the lung, ovarian, |
| | | anticancer | pancreas, thyroid, |
| | | immunologic | liver |
| ior-t1 | Center of Molecular | Murine monoclonal | T-cell lymphoma; |
| | Immunology, | antibody; anticancer | psoriasis; |
| | Havana, Cuba | immunologic; | rheumatoid arthritis |
| | | antipsoriatic; | |
| | | antiarthritic | |
| | | immunologic | |
| MDX-220 | Immuno-Designed | Chimeric monoclonal | Cancer: prostate, |
| | Molecules | antibody; anticancer | colorectal |
| | | immunologic | |
| MRA | Chugai | Chimeric monoclonal | Rheumatoid |
| | Pharmaceutical, | antibody; antiarthritic | arthritis; cancer, |
| | Tokyo, Japan | immunologic; | myeloma; Crohn |
| | | anticancer | disease; Castleman |
| | | immunologic; GI | disease |
| | | inflammatory and | |
| | | bowel disorders | |
| H-11 scFv | Viventia Biotech, | Humanized | Non-Hodgkin |
| | Toronto, Canada | monoclonal antibody; | lymphoma, |
| | | anticancer | melanoma |
| | | immunologic | |
| Oregovomab | AltaRex, Waltham, | Monoclonal | Cancer: ovarian |
| | MA | antibody, murine; | |
| | | anticancer | |
| | | immunologic; | |
| | | immunoconjugate | |
| huJ591 MAb, BZL | Millennium | Chimeric monoclonal | Cancer: prostate |
| | Pharmaceuticals, | antibody; anticancer | and general |
| | Cambridge, MA; | immunologic | |
| | BZL Biologics, | | |
| | Framingham, MA | | |
| Visilizumab | Protein Design Labs | Chimeric monoclonal | Transplant |
| | | antibody; | rejection, bone |
| | | immunosuppressant; | marrow; cancer, T- |
| | | anticancer | cell lymphoma; |
| | | immunologic; GI | ulcerative colitis; |
| | | inflammatory and | myelodysplastic |
| | | bowel disorders | syndrome; systemic |
| | | | lupus erythematosus |
| TriGem | Titan | Murine monoclonal | Cancer: melanoma, |
| | Pharmaceuticals | antibody; anticancer | small cell of the |
| | | immunologic | lung, brain |
| TriAb | Titan | Murine monoclonal | Cancer: breast, non- |
| | Pharmaceuticals | antibody; anticancer | small cell of the |
| | | immunologic | lung, colorectal |
| R3 | Center of Molecular | Chimeric monoclonal | Cancer: head and |
| | Immunology | antibody; anticancer | neck; diagnosis of |
| | | immunologic; | cancer |
| | | imaging agent; | |
| | | immunoconjugate | |
| MT-201 | Micromet, Munich, | Humanized | Cancer: prostate, |
| | Germany | monoclonal antibody; | colorectal, stomach, |
| | | anticancer | non-small cell of |
| | | immunologic | the lung |
| G-250, | Johnson & Johnson | Chimeric monoclonal | Cancer: renal |
| unconjugated | | antibody; anticancer | |
| | | immunologic | |
| ACA-125 | CellControl | Monoclonal | Cancer: ovarian |
| | Biomedical, | antibody; anticancer | |
| | Martinsried, | immunologic | |
| | Germany | | |
| Onyvax-105 | Onyvax, London, | Monoclonal | Cancer: colorectal; |
| | England | antibody; anticancer | sarcoma, general |
| | | immunologic | |
| Phase 1 | | | |
| CDP-860 | Celltech, Slough, | Humanized | Cancer: general; |
| | England | monoclonal antibody; | restenosis |
| | | anticancer | |
| | | immunologic; | |
| | | cardiovascular | |
| BrevaRex MAb | AltaRex | Murine monoclonal | Cancer: myeloma, |
| | | antibody; anticancer | breast |
| | | immunologic | |
| AR54 | AltaRex | Murine monoclonal | Cancer: ovarian |
| | | antibody; anticancer | |
| | | immunologic | |
| IMC-1C11 | ImClone Systems | Chimeric monoclonal | Cancer: colorectal |
| | | antibody; anticancer | |
| | | immunologic | |
| GlioMAb-H | Viventia Biotech | Humanized | Diagnosis of |
| | | monoclonal antibody; | cancer; cancer, |
| | | imaging agent; | brain |
| | | anticancer | |
| | | immunologic | |
| ING-1 | Xoma, Berkeley, | Chimeric monoclonal | Cancer: breast, lung |
| | CA | antibody; anticancer | (general), ovarian, |
| | | immunologic | prostate |
| Anti-LCG MAbs | eXegenics, Dallas, | Monoclonal | Cancer: lung, |
| | TX | antibody; anticancer; | general; diagnosis |
| | | imaging agent | of cancer |
| MT-103 | Micromet | Murine monoclonal | B-cell lymphoma, |
| | | antibody; anticancer | non-Hodgkin |
| | | immunologic | lymphoma, chronic |
| | | | myelogenous |
| | | | leukemia, acute |
| | | | myelogenous |
| | | | leukemia |
| KSB-303 | KS Biomedix, | Chimeric monoclonal | Diagnosis of |
| | Guildford, England | antibody; anticancer | cancer; cancer, |
| | | immunologic | colorectal |
| Therex | Antisoma, London, | Chimeric monoclonal | Cancer: breast |
| | England | antibody; anticancer | |
| | | immunologic | |
| KW-2871 | Kyowa Hakko, | Chimeric monoclonal | Melanoma |
| | Tokyo, Japan | antibody; anticancer | |
| | | immunologic | |
| Anti-HMI.24 | Chugai | Chimeric monoclonal | Myeloma |
| | | antibody; anticancer | |
| | | immunologic | |
| Anti-PTHrP | Chugai | Chimeric | Hypercalcemia of |
| | | monoclonal antibody; | malignancy; cancer, |
| | | anticancer | bone |
| | | immunologic; | |
| | | osteoporosis | |
| 2C4 antibody | Genentech | Chimeric monoclonal | Cancer: breast |
| | | antibody; anticancer | |
| | | immunologic | |
| SGN-30 | Seattle Genetics, | Monoclonal | Hodgkin lymphoma |
| | Seattle, WA | antibody; anticancer | |
| | | immunologic; | |
| | | multiple sclerosis | |
| | | treatment; | |
| | | immunosuppressant; | |
| | | immunoconjugate | |
| TRAIL-RI MAb, | Cambridge | Humanized | Cancer: general |
| CAT | Antibody | monoclonal antibody; | |
| | Technology, | anticancer | |
| | Cambridge, England | immunologic | |
| Prostate cancer | Biovation, | Monoclonal | Cancer: prostate |
| antibody | Aberdeen, Scotland | antibody; anticancer | |
| H22xKi-4 | Medarex | Chimeric monoclonal | Hodgkin lymphoma |
| | | antibody; anticancer | |
| | | immuologic | |
| ABX-MA1 | Abgenix | Humanized | Melanoma |
| | | monoclonal antibody; | |
| | | anticancer | |
| | | immunologic | |
| Imuteran | Nonindustrial | Monoclonal | Cancer: breast, |
| | source | antibody; anticancer | ovarian |
| | | immunologic | |

Clinical Trial

| | | | |
|---|---|---|---|
| Monopharm-C | Viventia Biotech | Monoclonal | Cancer: colorectal; |
| | | antibody; anticancer | diagnosis of cancer |
| | | immunologic; | |
| | | imaging agent | |

Methods for making suitable antibodies are known in the art. A full-length cancer-related antigen or antigenic peptide fragment thereof can be used as an immunogen, or can be used to identify antibodies made with other immunogens, e.g., cells, membrane preparations, and the like, e.g., E rosette positive purified normal human peripheral T cells, as described in U.S. Pat. No. 4,361,549 and 4,654,210.

Methods for making monoclonal antibodies are known in the art. Basically, the process involves obtaining antibody-secreting immune cells (lymphocytes) from the spleen of a mammal (e.g., mouse) that has been previously immunized with the antigen of interest (e.g., a cancer-related antigen) either in vivo or in vitro. The antibody-secreting lymphocytes are then fused with myeloma cells or transformed cells that are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference.

Mammalian lymphocytes are immunized by in vivo immunization of the animal (e.g., a mouse) with a cancer-related antigen. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by known techniques, for example, using polyethylene glycol (“PEG”) or other fusing agents (See Milstein and Kohler, Eur. J. Immunol. 6:511 (1976), which is hereby incorporated by reference). This immortal cell line, which is preferably murine, but can also be derived from cells of other mammalian species, including but not limited to rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also known. Typically, such antibodies can be raised by administering the protein or polypeptide of the present invention subcutaneously to New Zealand white rabbits that have first been bled to obtain pre-immune serum. The antigens can be injected at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthanized, e.g., with pentobarbital 150 mg/Kg IV. This and other procedures for raising polyclonal antibodies are disclosed in E. Harlow, et. al., editors, Antibodies: A Laboratory Manual (1988).

In addition to utilizing whole antibodies, the invention encompasses the use of binding portions of such antibodies. Such binding portions include F(ab) fragments, F(ab′)₂ fragments, and Fv fragments. These antibody fragments can be made by conventional procedures, such as proteolytic fragmentation procedures, as described in J. Goding, Monoclonal Antibodies: Principles and Practice, pp. 98-118 (N.Y. Academic Press 1983).

Examples of immunologically active portions of immunoglobulin molecules include F(ab) and F(ab′)₂ fragments, which retain the ability to bind antigen. Such fragments can be obtained commercially, or using methods known in the art. For example F(ab′)₂ fragments can be generated by treating the antibody with an enzyme such as pepsin, a non-specific endopeptidase that normally produces one F(ab′)₂ fragment and numerous small peptides of the Fc portion. The resulting F(ab′)₂ fragment is composed of two disulfide-connected F(ab) units. The Fc fragment is extensively degraded and can be separated from the F(ab)2 by dialysis, gel filtration, or ion exchange chromatography. F(ab) fragments can be generated using papain, a non-specific thiol-endopeptidase that digests IgG molecules, in the presence of a reducing agent, into three fragments of similar size: two Fab fragments and one Fc fragment. When Fc fragments are of interest, papain is the enzyme of choice, because it yields a 50,00 Dalton Fc fragment. To isolate the F(ab) fragments, the Fc fragments can be removed, e.g., by affinity purification using protein A/G. A number of kits are available commercially for generating F(ab) fragments, including the ImmunoPure IgG1 Fab and F(ab′)₂ Preparation Kit (Pierce Biotechnology, Rockford, Ill.). In addition, commercially available services for generating antigen-binding fragments can be used, e.g., Bio Express, West Lebanon, N.H.

Chimeric, humanized, de-immunized, or completely human antibodies are desirable for applications which include repeated administration, e.g., therapeutic treatment of human subjects.

Chimeric antibodies generally contain portions of two different antibodies, typically of two different species. Generally, such antibodies contain human constant regions and variable regions from another species, e.g., murine variable regions. For example, mouse/human chimeric antibodies have been reported which exhibit binding characteristics of the parental mouse antibody, and effector functions associated with the human constant region. See, e.g., Cabilly et al., U.S. Pat. No. 4,816,567; Shoemaker et al., U.S. Pat. No. 4,978,745; Beavers et al., U.S. Pat. No. 4,975,369; and Boss et al., U.S. Pat. No. 4,816,397, all of which are incorporated by reference herein. Generally, these chimeric antibodies are constructed by preparing a genomic gene library from DNA extracted from pre-existing murine hybridomas (Nishimura et al., Cancer Research, 47:999 (1987)). The library is then screened for variable region genes from both heavy and light chains exhibiting the correct antibody fragment rearrangement patterns. Alternatively, cDNA libraries are prepared from RNA extracted from the hybridomas and screened, or the variable regions are obtained by polymerase chain reaction. The cloned variable region genes are then ligated into an expression vector containing cloned cassettes of the appropriate heavy or light chain human constant region gene. The chimeric genes can then be expressed in a cell line of choice, e.g., a murine myeloma line. Such chimeric antibodies have been used in human therapy.

Humanized antibodies are known in the art. Typically, “humanization” results in an antibody that is less immunogenic, with complete retention of the antigen-binding properties of the original molecule. In order to retain all the antigen-binding properties of the original antibody, the structure of its combining-site has to be faithfully reproduced in the “humanized” version. This can potentially be achieved by transplanting the combining site of the nonhuman antibody onto a human framework, either (a) by grafting the entire nonhuman variable domains onto human constant regions to generate a chimeric antibody (Morrison et al., Proc. Natl. Acad. Sci., USA 81:6801 (1984); Morrison and Oi, Adv. Immunol. 44:65 (1988) (which preserves the ligand-binding properties, but which also retains the immunogenicity of the nonhuman variable domains); (b) by grafting only the nonhuman CDRs onto human framework and constant regions with or without retention of critical framework residues (Jones et al. Nature, 321:522 (1986); Verhoeyen et al., Science 239:1539 (1988)); or (c) by transplanting the entire nonhuman variable domains (to preserve ligand-binding properties) but also “cloaking” them with a human-like surface through judicious replacement of exposed residues (to reduce antigenicity) (Padlan, Molec. Immunol. 28:489 (1991)).

Humanization by CDR grafting typically involves transplanting only the CDRs onto human fragment onto human framework and constant regions. Theoretically, this should substantially eliminate immunogenicity (except if allotypic or idiotypic differences exist). However, it has been reported that some framework residues of the original antibody also need to be preserved (Riechmann et al., Nature 332:323 (1988); Queen et al., Proc. Natl. Acad. Sci. USA 86:10,029 (1989)). The framework residues which need to be preserved can be identified by computer modeling. Alternatively, critical framework residues may potentially be identified by comparing known antibody combining site structures (Padlan, Molec. Immun. 31(3):169-217 (1994)). The invention also includes partially humanized antibodies, in which the 6 CDRs of the heavy and light chains and a limited number of structural amino acids of the murine monoclonal antibody are grafted by recombinant technology to the CDR-depleted human IgG scaffold (Jones et al., Nature 321:522-525 (1986)).

Deimmunized antibodies are made by replacing immunogenic epitopes in the murine variable domains with benign amino acid sequences, resulting in a deimmunized variable domain. The deimmunized variable domains are linked genetically to human IgG constant domains to yield a deimmunized antibody (Biovation, Aberdeen, Scotland).

The antibody can also be a single chain antibody. A single-chain antibody (scFV) can be engineered (see, for example, Colcher et al., Ann. N.Y. Acad. Sci. 880:263-80 (1999); and Reiter, Clin. Cancer Res. 2:245-52 (1996)). The single chain antibody can be dimerized or multimerized to generate multivalent antibodies having specificities for different epitopes of the same target protein. In some embodiments, the antibody is monovalent, e.g., as described in Abbs et al., Ther. Immunol. 1(6):325-31 (1994), incorporated herein by reference.

Low Molecular Weight Targeting Ligands

Low molecular weight ligands, e.g., peptides and small molecules, with a molecular weight of less than about 2000, e.g., 1800, 1500, 1400, 1300, 1200, 1100 or less, e.g., 1000 can be used. Specific examples of low molecular weight peptides are peptides that bind specifically and preferentially to bladder cancer over normal bladder urothelial cells. Some amino acid sequences for bladder cancer-specific peptides are shown below in TABLE 4. The consensus peptide sequence is shown below each group.

Note that the first serine and the (glycine-serine)₄ spacer are from a phage display vector and are therefore invariant in all sequences. Invariant cysteine residues used to constrain peptide structure are shown in boldface. å=aliphatic residues. Ø=Phe or Trp. X=any amino acid.

TABLE 4

| | | Peptide Sequence |
|---|---|---|
| Structure | Clone #(s) | Unique Peptide |
| Heterocyclic | 1,2,8,9,10,11,14,16,17,18 | S I S L G C W G P F C (G S)₄ |
| | 3 | S V S L G C F G P W C (G S)₄ |
| | 4,19 | S I G L G C W G P F C (G S)₄ |
| | 5 | S V S L G C W G L F C (G S)₄ |
| | 7 | S V S L N C W G I A C (G S)₄ |
| | 12,20 | S M S L G C W G P W C (G S)₄ |
| | 13 | S I S L G C F G R F C (G S)₄ |
| | Consensus | å S L G C W G P ø C |
| Cyclic | 6 | S C V Y A N W R W T C (G S)₄ |
| | 15 | S C V Y S N W R W Q C (G S)₄ |
| | Consensus | C V Y x N W R W x C |

Linear, cyclic, or heterocyclic peptides, and modified peptides having a molecular weight less than 1100 have several desirable properties, including rapid biodistribution, excellent tissue/tumor penetration, and possibly oral availability. In addition, such low molecular weight peptides, e.g., aminobisphosphonates, e.g., pamidronate, often have a relatively short plasma half-life, e.g., ten minutes. Moreover, since these low molecular weight ligands are typically specific for extracellular epitopes, there is no requirement that the peptides be cell-permeable. Other specific low molecular weight peptides, namely, β-AG (13), and GPI-18648 (14) are shown in FIG. 7. Each peptide of FIG. 7 is a PSMA enzyme inhibitor.

In specific embodiments, low molecular weight ligands for making conjugates include pamidronate, GPI-18648 (FIG. 7), and ocreotide (sandostatin). To make the corresponding conjugate, the ligand is suspended in 100 μL of phosphate buffer with a pH of 7.4. A¹⁸FDGA-NHS (8) is eluted from a purification column that is similar to that described above in reference to FIGS. 5 and 5A, and approximately 400 μL is dripped directly into the ligand molecule suspension. Formation of conjugates proceeds at room temperature for twenty minutes until the reaction is quenched by addition of 100 mM Tris buffer (pH 8.5). The resulting molecules of formula (12′), are purified as described below, and then can be used for in vitro and in vivo imaging, e.g., PET imaging.

Synthetic Polymer Ligands

Polymers, e.g., synthetic polymers, can be used as ligands to form conjugates that are protected against rapid clearance from the body. For example, a polyol, e.g., a polyethylene glycol, a polypropylene glycol, and copolymers of a polyethylene glycol and a polypropylene glycol. Such glycols are available from BASF (Pluronic®) and Dow Chemical (Polyox®). These polymers can also be used in conjunction with targeting ligands to form protected, targeted conjugates.

Purification of Conjugates

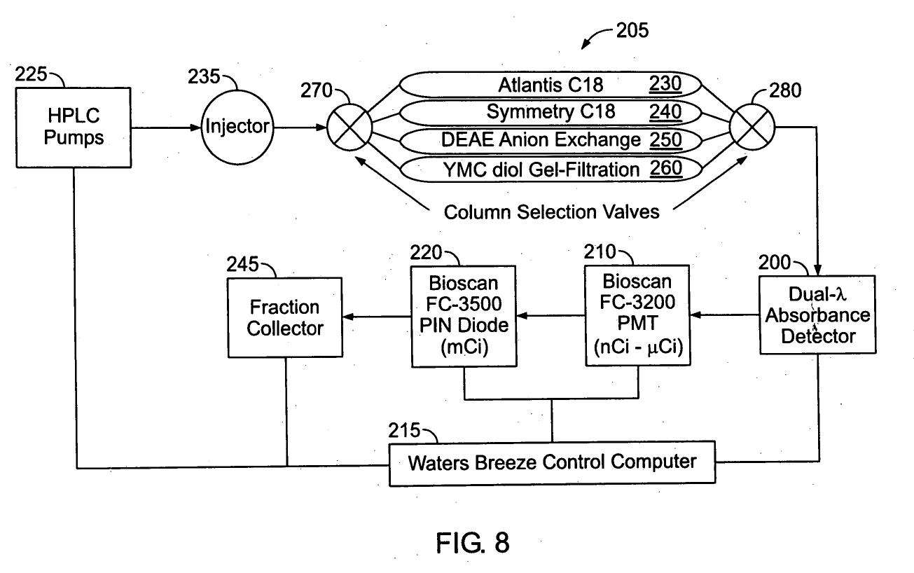

Purification of the conjugates can be performed, for example, using HPLC. Referring to FIG. 8, a series of detectors for absorbance 200, low-level gamma emission 210, and high-level gamma emission 220 can be used to ensure that all reaction products are detected and identified. The HPLC system 205 is controlled with a computer 215 that drives pumps 225, sequences an injector 235, and operates a fraction collector 245. The system is designed to have up to four different columns 230, 240, 250, and 260. Flow through columns 230, 240, 250, and 260 is controlled by selectable valves 270 and 280. For example, columns 230, 240, 250, and 260 can be, respectively, a Waters Atlantis™ C18 column, a Waters Symmetry® C18 column, a Nest DEAE column, and a Dionex YMC diol gel-filtration column.

For purifying the pamidronate conjugate of A¹⁸FDGA-NHS (8), DEAE anion exchange resin can be used, using a 0% A to 75% B gradient, where A=10 mM sodium phosphate at pH 7.4, and B=A+2 M NaCl. Under these conditions, the pamidronate conjugate should elute at approximately 45% B.

For purifying the GPI-18648 conjugate of A¹⁸FDGA-NHS (8), DEAE anion exchange resin is most appropriate, using a 0% A to 50% B gradient, where A=10 mM sodium phosphate at pH 7.4, and B=A+2 M NaCl. Under these conditions, the GPI-18648 conjugate should to elute at 30% B.

For purifying the MB-1 peptide conjugate of A¹⁸FDGA-NHS (8), a Symmetry C18 resin, using a 0% A to 100% B gradient can be used, where A=H2O+0.1% TFA, and B=acetonitrile+0.1% TFA. Under these conditions, the MB-1 peptide conjugate should elute at 60% B.

For purifying the Annexin conjugate of A¹⁸FDGA-NHS (8), a YMC diol gel filtration resin, using an isocratic PBS solutions at pH 7.4 can be used. Annexin conjugate is expected to elute in the void volume.

Applications

The ¹⁸F radio-labeled conjugates have a specific affinity for certain abnormal cells, e.g., cancer cells, and can be useful, e.g., in in-vivo pathology imaging, e.g., tumor imaging using PET. When properly configured, e.g., when R₁ of structure (12′) includes a molecular architecture that can bind specifically to a moiety of interest, the ¹⁸F radio-labeled conjugates can be used to specifically image abnormalities of the bladder, the brain, kidneys, lungs, skin, pancreas, intestines, uterus, adrenal gland, and eyes, e.g., retina.

¹⁸F conjugates will also find utility in other fields. For example, the annexin V derivative described above can be used to detect cell injury and death in the heart after a myocardial infarction. Moreover, ¹⁸F conjugates can be used to image non-cancerous cells in various tissues and organs under study, e.g., cells of the immune system. Imaging immune cells can aid in identifying sites of infection and inflammation.

### EXAMPLES

The invention is further described in the following examples, which do not limit the scope of the invention described in the claims.

Materials

2-deoxy-2-[¹⁸F]fluoro-D-glucose, ¹⁸FDG (1), was obtained as 55 nM (10 mCi) aqueous solution from either Cardinal Health or PETnet. Bromine, NHS, dimethoxymethane, ascorbic acid and EDC were obtained from Aldrich Chemical, and were used as received.

### Example 1

### Mass Spectroscopic Identification of Intermediates

Electrospray mass spectrometry was used to analyze ¹⁸FDG (1), and some of the radio-labeled derivatives shown in FIG. 3. The spectrometer was a Waters LCT Hexapole Electrospray time-of-flight mass spectrometer, and was operated in positive ion mode, using ammonium acetate as carrier.

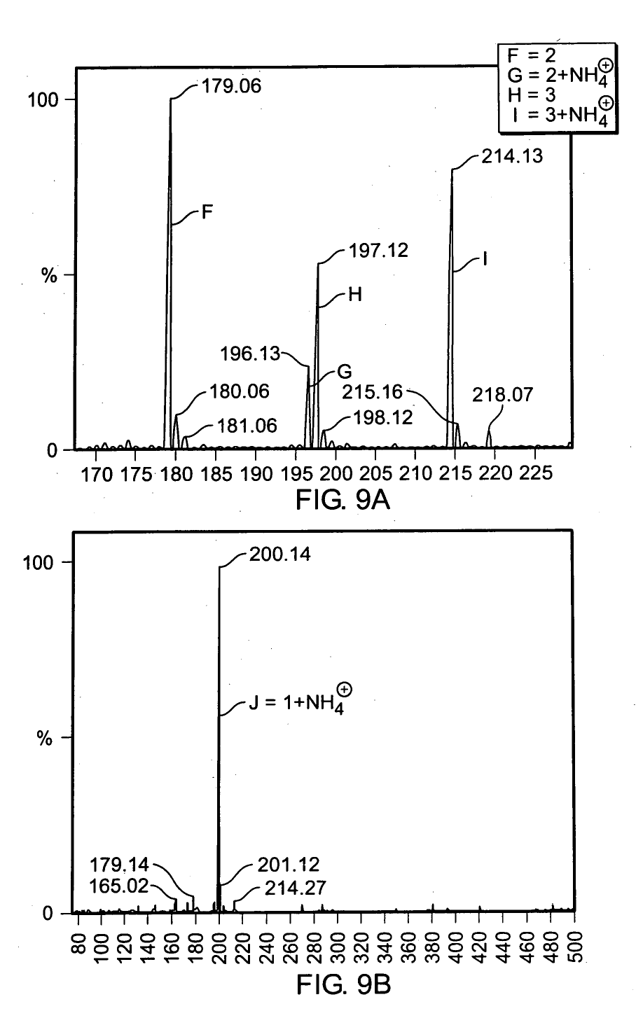

FIG. 9A shows a mass spectrum that has a peak (F) for compound (2), and a peak (G) for its ammonium adduct, which has a mass of (2)+NH₄⁺. In addition, the mass spectrum show has a peak (H) for compound (3), and a peak (I) for its ammonium adduct, which has a mass of (3)+NH₄⁺. FIG. 9B has a peak (I) for the ammonium adduct of ¹⁸FDG (1), which has a mass of (1)+NH₄⁺. Together, FIGS. 9A and 9B show that electrospray mass spectrometry is a convenient method for analyzing compositions of ¹⁸FDG (1), and some its radio-labeled derivatives.

### Example 2

### HPLC Separation and Purification of Succinimidyl Esters

HPLC was used to analyze some of the radio-labeled ¹⁸FDG derivatives shown in FIG. 3. Evaporative light scattering detection (ELSD) was used for peak detection. Separation was achieved with a Waters Atlantis™ C18 column, and detection of eluant was achieved with a Sedex Model 75 ELSD. This particular ELSD detector has a sensitivity of less than 10 ng for sugars, such as glucose and ¹⁸FDG.

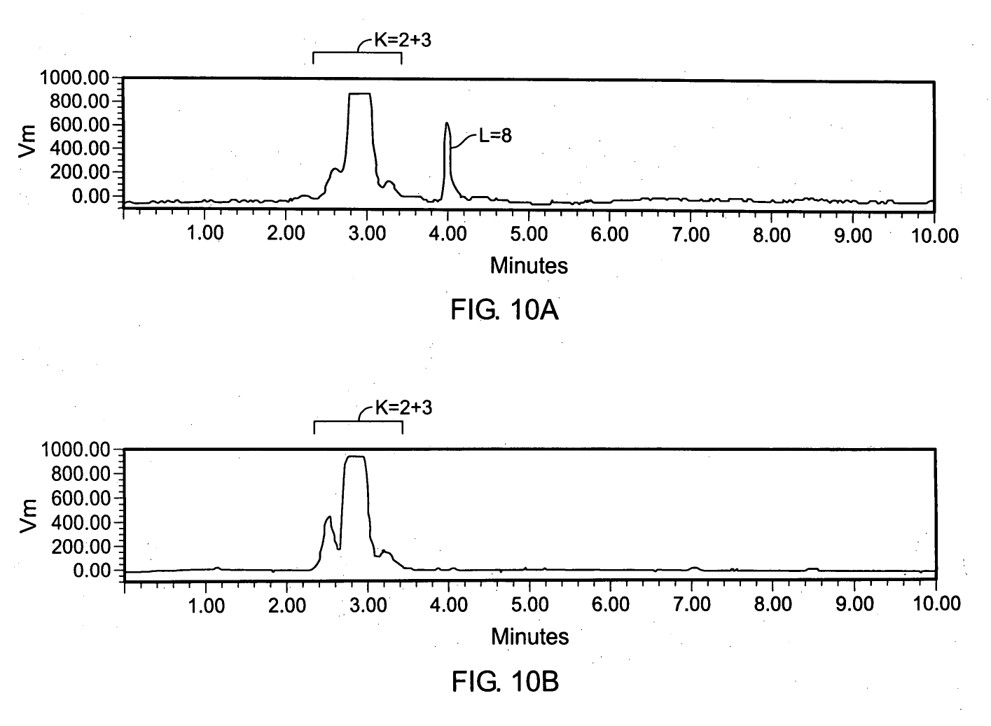

A solution containing gluconic acid (3) and its lactone (2) was protected with dimethoxymethane. Excess bromine was quenched with ascorbic acid. To this resulting solution was added EDC and NHS in MES buffer at pH 5.5. After 2 hours, the reaction mixture was diluted and separated on an Atlantis C18 column using an isocratic mobile phase of H₂O+0.1% trifluoroacetic acid. FIG. 10A shows an HPLC trace that includes a region (K) that is a mixture of compounds (2) and (3), and a region (L) that is compound (8). By comparison, FIG. 10B shows a control chromatogram of a mixture of the gluconic acid (3) and its lactone (2) in MES buffer.

Together, FIGS. 10A and 10B show that HPLC is a convenient method for analyzing compositions of ¹⁸FDG (1) derivatives, to separate, purify, and detect the succinimidyl ester (8).

### Example 3

### Three-Dimensional PET Imaging

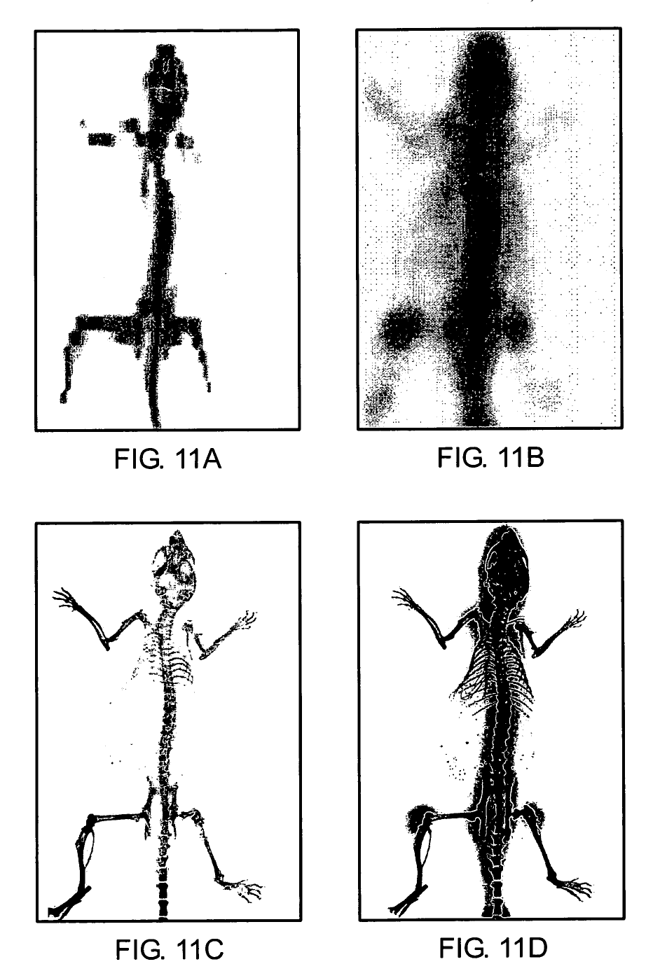

A GE Discovery LS PET/CT scanner can be used to scan animals, e.g., humans. Small animals, e.g., mice, can also be scanned by combining data sets from the Discovery LS, and a GE Explore RS micro-CT, e.g., to optimize conjugates for a particular application (see FIGS. 11A-11D). Several mice, can be imaged simultaneously using a holder with nine “tubes.”

FIG. 11A is a CT data set from a human PET/CT, while FIG. 11B is a PET data set from a human PET/CT. FIG. 11C is a micro-CT data set from a GE Explore RS. Data sets of FIGS. 11A and 11B are automatically co-registered by the Discovery LS. After co-registration of the data sets of FIGS. 11A and 11C, the data set of FIG. 11A is deleted, resulting in the data set presented in FIG. 11D, which is a fusion of micro-CT and clinical PET data sets. This technique permits PET imaging of small animals on a human scanner. In this Example, 750 μCi of ¹⁸F-NaF was injected into the tail vein of a 25 g CD-1 mouse. The mouse was imaged 90 minutes later.

### Example 4

### Conjugate of Pamidronate

To make the conjugate, pamidronate is suspended in 100 μL of phosphate buffer with a pH of 7.4. A¹⁸FDGA-NHS (8) is eluted from a purification column that is similar to that described above in reference to FIGS. 5 and 5A, and approximately 400 μL is dripped directly into the ligand molecule suspension. Formation of conjugates proceeds at room temperature for twenty minutes until the reaction is quenched by addition of 100 mM Tris buffer (pH 8.5).

### Example 5

### Conjugate of GPI-18648

To make the conjugate, GPI-18648 is suspended in 100 μL of phosphate buffer with a pH of 7.4. A¹⁸FDGA-NHS (8) is eluted from a purification column that is similar to that described above in reference to FIGS. 5 and 5A, and approximately 400 μL is dripped directly into the ligand molecule suspension. Formation of conjugates proceeds at room temperature for twenty minutes until the reaction is quenched by addition of 100 mM Tris buffer (pH 8.5).

### Example 6

### Conjugate of Ocreotide (Sandostatin)

To make the conjugate, ocreotide is suspended in 100 μL of phosphate buffer with a pH of 7.4. A¹⁸FDGA-NHS (8) is eluted from a purification column that is similar to that described above in reference to FIGS. 5 and 5A, and approximately 400 μL is dripped directly into the ligand molecule suspension. Formation of conjugates proceeds at room temperature for twenty minutes until the reaction is quenched by addition of 100 mM Tris buffer (pH 8.5).

### Other Embodiments

A number of embodiments of the invention have been described. Nevertheless, it will be understood that various modifications may be made without departing from the spirit and scope of the invention.

What is claimed is:

1. A method of making a 2-deoxy-2-[¹⁸F]fluoro-D-glucose derivative, the method comprising:
oxidizing ¹⁸FDG with an oxidant under first conditions and for a sufficient first time to produce a gluconic acid lactone that is in equilibrium with its gluconic acid form;
protecting the gluconic acid form by reacting two hydroxyl groups of the gluconic acid form with a protecting moiety under second conditions and for a sufficient second time to prevent reversion of the gluconic acid form to its gluconic acid lactone, and to produce a protected acid the protected acid having a carboxylic acid group that includes a carboxylic acid hydroxyl group; and
substituting the carboxylic acid hydroxyl group of the protected acid with a leaving group (LG), thereby forming an ¹⁸FDG derivative.

2. The method of claim 1, wherein the ¹⁸FDG derivative is a compound of formula (5) 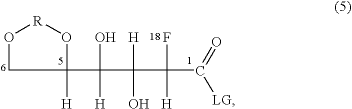
wherein LG and R each, independently, comprises an alkyl group, an alkenyl group, an alkynyl group, an aryl group, a heterocyclic group, a boron-containing group, or a mixture of such groups, and wherein LG and R each comprise no more than twenty carbon atoms.

3. The method of claim 1, wherein the two reacted hydroxyl groups are located on adjacent carbons.

4. The method of claim 1, wherein the oxidant is diatomic bromine.

5. The method of claim 1, wherein the first conditions includes use of a buffer solution.

6. The method of claim 1, wherein the buffer solution comprises a phosphate buffer.

7. The method of claim 1, wherein the first conditions include maintaining a pH of about 4 to about 9.

8. The method of claim 1, wherein the first conditions include maintaining a temperature from about 15 to about 50° C.

9. The method of claim 1, wherein the second conditions include maintaining a pH of about 0 to about 5.

10. The method of claim 1, wherein the second conditions include maintaining a temperature from about 15 to about 60° C.

11. The method of claim 1, wherein the two hydroxyl groups are attached to C5 and C6, or C4 and C5, or C4 and C6 of formula (3):

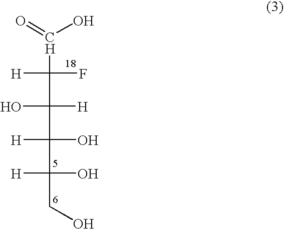

12. The method of claim 1, wherein the protecting moiety is selected from the group consisting of formaldehyde, dimethoxymethane, boric acid, and mixtures thereof.

13. The method of claim 1, wherein the leaving group is O—N-succinimide.

14. A compound of formula (5) 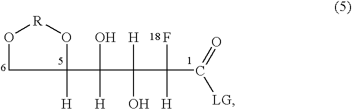
wherein LG and R each, independently, comprises an alkyl group, an alkenyl group, an alkynyl group, an aryl group, a heterocyclic group, a boron-containing group, or a mixture of such groups, and wherein LG and R each comprise no more than twenty carbon atoms.

15. The compound of claim 14, wherein LG is O—N-succinimide, and wherein R is (CH₂)ₙ, n being an integer between 1 and 10, inclusive.

16. The compound of claim 14, wherein LG is O—N-succinimide, and wherein R is CH₂.

17. A compound of formula (4) 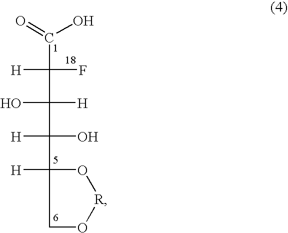
wherein R comprises an alkyl group, an alkenyl group, an alkynyl group, an aryl group, a heterocyclic group, a boron-containing group, or a mixture of such groups, and wherein R comprises no more than twenty carbon atoms.

18. The compound of claim 17, wherein R is (CH₂)ₙ, n being an integer between 1 and 10, inclusive.

19. A method of purifying a radio-labeled 2-deoxy-2-[¹⁸F]fluoro-D-glucose derivative, the method comprising:
obtaining a composition comprising (¹⁸FDG), a solvent, and a compound of claim 18, wherein LG and R each, independently, comprises an alkyl group, an alkenyl group, an alkynyl group, an aryl group, a heterocyclic group, a boron-containing group, or a mixture of such groups, and wherein LG and R each comprise no more than twenty carbon atoms;
flowing the composition through a column that comprises an adsorbent, the absorbent binding to the compound of formula (5) with a greater affinity than other components of the composition; and
eluting the compound of formula (5), substantially free ¹⁸FDG

20. The method of claim 19, wherein the compound of formula (5) is A¹⁸FDGA-NHS.

21. The method of claim 19, wherein the adsorbent is a resin

22. The method of claim 21, wherein the resin is cross-linked.
